(19) United States
(12) Patent Application Publication
(10) Pub. No.: US 2026/0308619 A1
(43) Pub. Date: Oct. 8, 2026

(54) RETROVIRAL VECTORS

(71) Applicant: UCL BUSINESS LTD, London (GB)

(72) Inventors: John COUNSELL, London (GB); Antonio GRECO, London (GB); Zeinab ASGARIAN, London (GB); Ala'a SIAM, London (GB); Luke TAPPOUNI, London (GB)

(21) Appl. No.: 19/480,533

(22) PCT Filed: May 3, 2024
(86) PCT No.: PCT/GB2024/051177

§ 371 Date: Oct. 31, 2025

(30) Foreign Application Priority Data
May 3, 2023 (GB) 2306491.8

(51) Int. Cl. C12N 15/86; A61K 45/06; A61K 48/00

(52) U.S. Cl. CPC C12N 15/86; A61K 45/06; A61K 48/0033; A61K 48/0075; C12N 2740/16032; C12N 2740/16043; C12N 2830/42; C12N 2830/48; C12N 2830/50

(57) ABSTRACT

This invention relates to a novel retroviral vector comprising a 5′ long terminal repeat (LTR), a primer binding site, a heterologous intron, a transgene insertion site and a 3′ LTR, wherein the heterologous intron is located immediately downstream of the 5′ LTR. Such vectors have high titre production rates and are particularly suited for gene therapy.

### FIELD OF THE INVENTION

The present invention relates to modified lentiviral gene therapy vectors that minimise the production of multiple subgenomic RNAs during vector production in a target cell, enhance vector titres and have an improved safety profile. Such vectors have particular use as gene therapy vectors targeting the liver.

### BACKGROUND TO THE INVENTION

Retroviridae is a family of single-stranded RNA viruses. These viruses became attractive as vectors for gene therapy due to their large cloning capacity and ability to stably integrate virally encoded genomic information into the DNA of the host. Lentivirus is a genus of retrovirus derived from the human immunodeficiency virus type 1 (HIV-1). As with other retroviruses, lentiviruses reverse-transcribe their RNA genome into DNA, which is permanently integrated into the host genome. This makes them advantageous as viral vectors as there is persistent expression of the virally delivered gene of interest in the host cells, in contrast to adeno-associated viral vectors, which are rapidly depleted from dividing cells and thus require frequent administration. Lentiviral vectors also offer a greater range of therapeutic uses compared to other retroviral derived vectors as they can transduce both dividing and non-dividing cells.

Despite these advantages, the permanent integration of the lentiviral genome poses the risk of insertional mutagenesis. The persistence of cis elements within the transgene expression cassette, in particular the RNA packaging sequences such as the RNA packaging signal (ψ) and the Rev Response Element (RRE), can lead to interactions between the host and viral genomes. Splice sites within the cis elements have been show to splice with nearby host genes, resulting in aberrant fusion transcripts. These cis element sequences are also rich in CpG islands, which undergo DNA methylation in host cells, thus resulting in transgene silencing, and enable remobilization of lentiviral genomes in cells expressing lentiviral proteins. This can be problematic for HIV-positive patients. Therefore, it is necessary to minimise the persistence of these cis elements to improve the safety of lentiviral vectors in clinical translation.

Previous attempts to minimise insertional mutagenesis caused by lentiviral vectors have involved truncating the w and the RRE sequences or removing these RNA packaging sequences from the lentiviral vector provirus by Cre-loxP mediated deletion. However, these vectors have been limited by inefficient vector production and low titres.

WO 2015/056014 discloses a retroviral vector comprising a primer binding site, a long terminal repeat and an RNA packaging sequence, wherein the RNA packaging sequence is located 3′ of the long terminal repeat and no long terminal repeat is located 3′ of the RNA packaging sequence, such that reverse transcription initiated at the primer binding site does not lead to reverse transcription of the RNA packaging sequence into vector DNA in a target cell. The elimination of HIV-1 packaging sequences from lentiviral vector proviruses has been previously shown to enhance safety and to expedite gene transfer for gene therapy (4). However, the vector titre in both instances was low due to inefficient production of vector genomic RNA and the production of multiple subgenomic RNAs during vector production.

It is the object of the present invention to address one or more issues associated with the prior art retroviral vectors for use in gene therapy. In particular, it is an object of the present invention to provide a retroviral vector, which has a reduced production of subgenomic RNA during vector production and/or increased vector titres. It is also an object of the present invention to provide a lentiviral vector, which has increased vector titres whilst maintaining a low frequency of aberrant fusion transcripts in cells exposed to the vector during gene therapy.

### SUMMARY OF THE INVENTION

In accordance with a first aspect of the present invention, there is provided a retroviral vector comprising a 5′ long terminal repeat (LTR), a primer binding site (PBS), a heterologous intron, a transgene insertion site, and a 3′ LTR, wherein the heterologous intron is located immediately downstream of the 5′ LTR.

A primer binding site (PBS) will preferably be located between the 5′ LTR and the intron. The PBS is a site which binds to a tRNA primer, which is responsible for initiating minus strand synthesis during the reverse transcription process.

In some embodiments, the retroviral vector comprises a primer having a primer over-extension sequence (POS). The POS is a conserved motif that is adjacent to and downstream of the primer binding site. It comprises the sequence [C/T] TGAAA and provides a region of complementarity for over-extended products therefore enabling their participation in the plus strand transfer reaction. The primer over-extension sequence is adjacent to and immediately upstream of the intron.

Retroviral LTRs are generally segmented into U3, R and U5 regions. However, in certain LTRs, parts of these regions may be deleted. The term “long terminal repeat” or “LTR” is intended to cover all such variations. The LTR participates in the reverse transcription process so that vector DNA is produced in the target cell based on the vector RNA. LTRs can comprise a number of signals required for gene expression, such as a transcriptional enhancer, a promoter, a transcription initiation signal and/or a polyadenylation signal.

The 5′ LTR may comprise an HIV-1 LTR or derivative sequence thereof. The 5′ LTR has the sequence of SEQ ID No. 2 or a derivative sequence having at least about 40% identity thereof.

In some embodiments, if the 5′ LTR sequence is a derivative sequence, the derivative sequence may have at least about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, or about 99% sequence identity thereof to SEQ ID No. 2.

For all aspects, sequence identity is determined by comparing two aligned substantially complementary sequences over their length and overall identity is expressed as a percentage. The measurement of nucleotide sequence identity is well known in the art, using specialist computer programs such as “BLAST”.

The 3′ LTR preferably comprises a HIV-1 LTR or derivative sequence thereof. In one embodiment, the 3′ LTR sequence has the sequence of SEQ ID No. 8 or a derivative sequence having at least 40% identity thereof. The 3′ LTR comprises a polyadenylation (polyA) site in the R domain and a HIV-1 transacting responsive (TAR) hairpin site. In certain embodiments, the 3′ LTR may have a mutation in the polyA site so as to eliminate its activity (ApA) and/or a mutation in the TAR hairpin site (ATAR).

In some embodiments, if the 3′ LTR sequence is a derivative sequence, the derivative sequence may have at least about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, or about 99% sequence identity thereof to SEQ ID No. 8.

In one embodiment, the 3′ LTR may comprise a self-inactivating (SIN) LTRs. For enhanced safety, the 3′ LTR in the vector may preferably be a self-inactivating LTR, in which nucleotides in the U3 region have been deleted. This can include the TATA box and binding sites for transcription factors. The deletion is transferred to the 5′ LTR after reverse transcription in target cells, resulting in transcriptional inactivation of the LTR in the proviruses. SIN LTRs are well known to those skilled in the art (e.g see Retroviruses. Edited by Coffin JM, Hughes SH and Varmus HE. Cold Spring Harbor (NY): Cold Spring Harbor Laboratory Press; 1997).

Surprisingly, and advantageously, the inventors have found that insertion of a heterologous intron immediately downstream to the 5′ LTR increases the efficacy and safety profile of these vectors compared to conventional third-generation lentiviral vectors. The inventors unexpectedly found that a lower dose of the vector of the present invention could be used to achieve the same efficacy as previous generation CCL vectors. Without wishing to be bound by theory, the inventors believe that the lower dose will improve the safety as the vectors are less likely to induce a strong immune response and results in fewer integration events, therefore minimising possible disruption to the host genome.

In some embodiments, the intron may comprise the intron of the elongation factor 1a gene. In other embodiments the intron may comprise the intron of β-globin gene and/or a truncated version. In an alternative embodiment, the intron may comprise the intron of the chicken beta actin (CBA) gene. Preferably, the intron comprises a sequence in the range of about 100 to about 2,000 base pairs. More preferably, the intron comprises a sequence in the range of about 200 to about 1,000 base pairs.

The vector may further comprise a HIV-1 psi packaging sequence and/or Rev Response Element (RRE) located downstream of the 3′ LTR.

The vector may comprise further elements, which help in transduction and expression of the vector. These elements are generally located between the PBS and the 3′ LTR. For example, the vector may further comprise a post-transcriptional regulatory element (PRE), such as a woodchuck hepatitis virus post-transcriptional regulatory element (WPRE).

The vector may further comprise a central polypurine tract (cPPT). This is a region used as a primer to synthesise the plus strand during retroviral replication. Preferably this is located downstream of the intron. Preferably this is located between the intron and the transgene promoter, in which the promoter is downstream of the cPPT. The vector may also comprise a central termination site (CTS).

The vector preferably comprises a promoter to drive transcription of the vector genome. This may be any suitable promoter, including a Rous Sarcoma Virus (RSV) promoter, a human cytomegalovirus (CMV) immediate early promoter, a spleen focus forming virus (SFFV) promoter, or an HIV-1 U3 promoter. This promoter is preferably positioned at the 5′ end of the vector.

Preferably, the primer binding site of the vector is positioned precisely on the transcription start site of the promoter, which drives transcription of the vector genome.

The elements will preferably comprise:

| SEQ | |
|---|---|
| ID | |
| NO. | Description |
| 1 | Cytomegalovirus (CMV) promoter |
| 2 | 5′ long terminal repeat (LTR) |
| 3 | Primer binding site (PBS) |
| | Primer overextension sequence |
| 4 | Elongation factor 1α intron (EF1A) |
| 5 | β globin intron |
| 6 | Central polypurine tract (cPPT) and central termination site (CTS) |
| 7 | Woodchuck Hepatitis Virus (WPRE) |
| 8 | 3′ self-inactivating long terminal repeat (SIN-LTR) |
| 9 | 3′ long terminal repeat (LTR) with polyadenylation (polyA) site |
| | mutation |
| 10 | 3′ long terminal repeat (LTR) with HIV-1 transacting responsive |
| | (TAR) hairpin site mutation |
| 11 | 3′ long terminal repeat (LTR) with polyadenylation (polyA) site |
| | mutation and HIV-1 transacting responsive (TAR) hairpin hairpin |
| | site |
| 12 | HIV-1 psi packaging sequence |
| 13 | Rev Response Element (RRE) |
| 14 | ΔGag-RRE |

The retroviral vector may further comprise an expressible transgene, or multiple transgenes, in the transgene insertion site.

In some embodiments, the transgene is a non-retroviral gene and may be any gene of which is desired in a target cell. The transgene should be located between the PBS and the 3′ LTR. The transgene may encode for a peptide or protein to a noncoding RNA. In some embodiments, the transgene encodes for a peptide or protein. Preferably, the peptide or protein should be useful in gene therapy. In some embodiments, the transgene is under the control of a promoter (e.g. a PGK, LP1 or GADPH promoter). It will be apparent to the skilled addressee that a number of different promoters can be employed.

Expression of the transgene may aid normal growth of the cell or maintain the health of a subject. In some embodiments, the transgene encodes for a peptide or protein, which is absent or under expressed in a subject. Alternatively, the transgene may encode for a peptide or protein, which helps to prevent or ameliorate a medical condition. The peptide or protein may be one, which is useful in treating diseases, such as cancer, atherosclerosis, sickle-cell anaemia, infection, metabolic disorders, neurological illness, and thalassemia. Examples of such peptides and proteins are haemoglobin, hematopoietic growth factors, such as granulocyte-macrophage colony stimulating factor (GM-CSF), macrophage colony stimulating factor (M-CSF), granulocyte colony stimulating factor (G-CSF), erythropoietin (EPO), common gamma chain, Wiskott Aldrich Syndrome protein (WASp), GP91phox, and ABCD1. Another example is tumour necrosis factor (TNF), which is a molecule that can be used to treat cancer and, in particular, tumours. The tumour suppressor factor p53 and retinoblastoma (RB) are also contemplated. Various cytokine, such as mast cell growth factor (MGF) and interleukins 1-11 are also proteins, which are contemplated by the present invention. A multidrug resistance gene (mdR) encoding a p-glycoprotein is also contemplated as the transgene. The peptide or protein may also be a selectable marker for antibiotic resistance in eukaryotes. Other types of selectable markers, such as adenine phosphoribosyl transferase (APRT) in APRT-deficient cells, a fluorescence protein, or the firefly luciferase gene, are also included. The peptide or protein can be a protein that will provide the host with an additional or altered enzymatic activity, such as the herpes simplex virus thymidine kinase protein for ‘suicide therapy’ of reactive transplants, or a toxin, such as diphtheria toxin protein for a treatment of cancer. The transgenes encoding these proteins can be provided by any variety of methods, such as routine cloning procedures (Sambrook et al (1989), Molecular Cloning: A Laboratory Manual, Cold Spring Harbor, NY), excision from a vector containing the gene of interest, or chemical or enzymatic synthesis based on published sequence information. In many instances, the DNA encoding the protein of interest is commercially available. In another embodiment, the transgene encodes a protein, which enables experimental manipulation of the cell, for example a toxin or a fluorescent or drug-selectable marker.

In another preferred embodiment, the transgene is capable of being transcribed into a noncoding RNA molecule. In some embodiments, the transgene may encode a noncoding RNA, which can alter the level of expression of genes within the cell or is a component of a ribonucleoprotein complex with enzymatic activity. Examples of such noncoding RNAs are short interfering RNAs (siRNAs), microRNAs, small nucleolar RNAs (snoNAs), small nuclear RNAs (snRNAs), piwi interacting RNAs (piRNAs), long noncoding RNAs, transfer RNAs (tRNAs) and ribosomal RNAs (rRNAs). In some embodiments, the transgene may encode a noncoding RNA, which is sufficiently complementary to hybridise to an mRNA or DNA of interest. Such an RNA molecule is an antisense RNA and has utility in preventing or limiting the expression of over-produced, defective or otherwise undesirable molecules or to investigate the function of a gene. The vector of the present invention can comprise, as the transgene, a sequence encoding an antisense RNA, which is sufficiently complementary to a target sequence, such that it binds to the target sequence. For example, the target sequence can be part of the mRNA encoding a polypeptide such that it binds to and prevents translation of mRNA encoding the polypeptide. In another embodiment, the target sequence is a segment of a gene that is essential for transcription, such that the antisense RNA binds the segment (e.g., a promoter or coding region) and prevents or limits transcription. Hence, the antisense RNA must be of sufficient length or complementarity to prevent translation of its target mRNA or transcription of its target DNA. One of ordinary skill in the art can determine antisense molecules having sufficient complementarity to a target sequence, such that the antisense molecule is capable of binding to the target and thereby inhibiting translation or transcription. The transgene sequence can be provided, for example, by chemical or enzymatic synthesis, or from commercial sources.

The vector may further comprise a post-transcriptional regulatory element (PRE). The PRE may be located between the transgene insertion site and the 3′ LTR.

The retroviral vector can be based on any suitable retrovirus, which is able to deliver genetic information to eukaryotic cells. For example, the retroviral vector may be an alpharetroviral vector, a gammaretroviral vector, a lentiviral vector or a spumaretroviral vector. Such vectors have been used extensively in gene therapy treatment and other gene delivery applications. In some embodiments, the retroviral vector is a lentiviral vector. In some instances, the retroviral vector may be based on HIV-1. Preferably, the vector is derived from a lentivirus.

The vector comprises an RNA packaging sequence, which is located downstream of the 3′ LTR such that the RNA packaging sequence is not reverse transcribed into vector DNA in a target cell. The RNA packaging sequence is necessary for the essential process of packaging the retroviral RNA genome into the viral particle as it is assembled by the producer cell. The RNA packaging sequence is able to bind to viral proteins within the nascent viral particle. In some embodiments, the RNA packaging sequence comprises the RNA packaging signal (ψ). The RNA packaging signal may comprise the HIV-1 psi packaging sequence. In HIV-1, a portion of the gag gene has been found to be involved in RNA packaging. In some embodiments, a point mutation is introduced downstream of the gag start codon to prevent translation of the majority of this signal (AGag). The RNA packaging sequence may also comprise the Rev Response Element (RRE). The RNA packaging sequence may comprise AGag-RRE. AGag-RRE comprises the psi-packaging sequence, the RRE and parts of the retroviral env and gag genes. RNA packaging sequences and components that make this up are well known to those skilled in the art (e.g see Retroviruses. Edited by Coffin JM, Hughes SH and Varmus HE. Cold Spring Harbor (NY): Cold Spring Harbor Laboratory Press; 1997).

In some embodiments, the vector further comprises an exogenous nucleotide sequence for delivery into a target cell. This exogenous nucleotide sequence may be any sequence, which someone might want to insert into a target cell. For example, the exogenous nucleotide sequence may be an expressible transgene, an RNA interference cassette, or a molecular barcode, for example, for marking the lineage of different cells. The exogenous nucleotide sequence should be located between the PBS and the 3′ LTR.

In accordance with another aspect of the present invention, there is provided a host cell containing the vector as herein above described.

The host cell may be any suitable eukaryotic cell into which the vector may be introduced.

Plasmids encoding the retroviral vectors of the present invention are transfected into suitable host cells (or packaging cells) by standard methods known to one of ordinary skill in the art. Suitable packaging cells are defined herein as cells that contain helper virus sufficient to allow the packaging of RNA transcribed from the retroviral vector and the release of vector virus particles, or virions. Generally, additional plasmids encoding transacting viral sequences but lacking the cis-acting sequences required for packaging are co-transfected. These supply the required structural and enzymatic proteins to package and produce the transcribed viral genome. Such packaging cells are known and available to one of ordinary skill in the art, and include, for example, HEK293T cells.

Recombinant retrovirus produced from the transfected cells is harvested by standard methods. The harvested retrovirus, in the form of virions, is used to transduce a permissive target cell by standard techniques. A target cell is defined herein as any cell that is permissive to infection by the virus produced by the retroviral vector of the present invention. The target cell can be in vivo or ex vivo. Representative target cells include, for example, bone marrow stem cells, hepatocytes, muscle cells, tumour cells, neurons, retina, and airway epithelial cells. The provirus that is formed in the target cell can then express the transgene. Because the provirus contains no RNA packaging sequence, any endogenous helper proteins present cannot trigger production of an infectious virus from any provirus.

In accordance with another aspect of the present invention, there is provided a virion containing the vector as herein above described.

The retroviral vector or virion may be for use as a medicament.

The retroviral vector or the virion may be for use in the treatment of a disease requiring gene therapy.

In certain embodiments, the retroviral vector or virion is for administration to the liver. The retroviral vector or virion may be used to treat a number of diseases, such as metabolic disorders and/or hepatic diseases.

In accordance with a further aspect, there is provided pharmaceutical composition comprising the vector or virion.

The pharmaceutical composition may further comprise one or more pharmaceutically acceptable excipients.

The composition may be a liquid or a solid, for example a powder, gel, or paste. Preferably, a composition is a liquid, preferably an injectable liquid. Such an injectable liquid will preferably be suitable for hepatic artery infusion administration. The composition may also comprise one or more excipients and such excipients will be known to persons skilled in the art.

The composition may incorporate or be administered in conjunction (either sequentially or simultaneously) with (or co-administered with) an immunosuppressant. Such immunosuppressants may be selected from one or more of the following: tacrolimus, mycofenolate mofetil and prednisolone. The skilled addressee will understand that other immunosuppressants may also be employed.

The vector or virion may be for use in delivering a transgene to a subject with gene therapy.

In accordance with another aspect, there is provided a method of delivering a gene to a target cell, the method comprising administering an effective amount of the vector or virion to the target cell, wherein the vector or virion comprises an expressible transgene. This method can be used to create a cell line, which expresses the gene of interest. For example, the gene can encode for a biotherapeutic so that the cell line produces the biotherapeutic, for example, a therapeutic protein or antibody.

In accordance with another aspect of the present invention, there is provided a cell produced by the method.

In accordance with a yet further aspect of the present invention, there is provided a transgenic animal produced by the method.

Features, elements, characteristics, compounds, molecules, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and figures), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

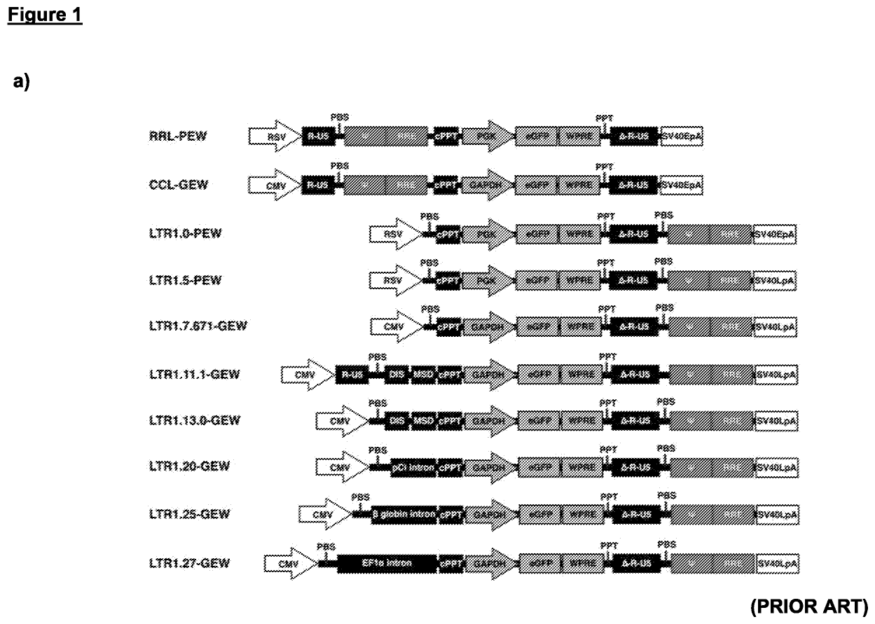

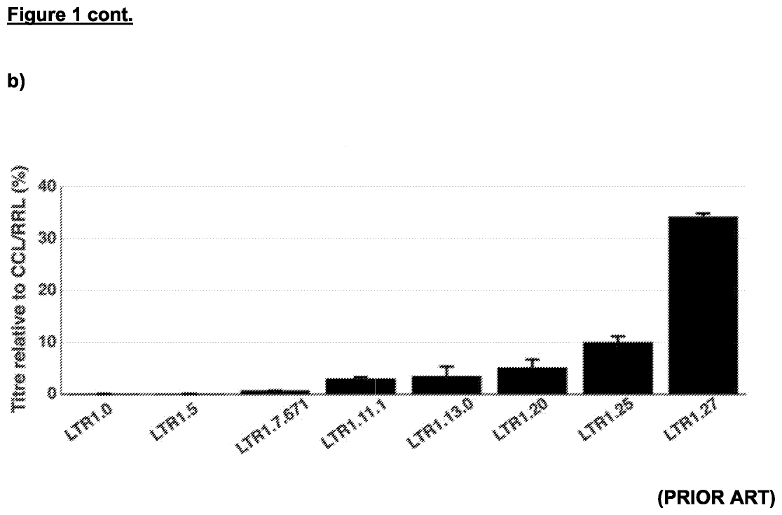

FIG. 1 is a schematic diagram and a graph taken from (4) showing the LTR1 structures developed based on the conventional previous generation lentiviral vectors, and their respective vector titres in comparison (prior art). (a) is a vector schematic diagram showing the modification of the LTR1 backbone. (b) is a graph showing the EGFP titres relative to the CCL/RRL vectors (%) generated with each of the vectors. The bars represent mean values with error bars showing standard error of the mean (SEM).

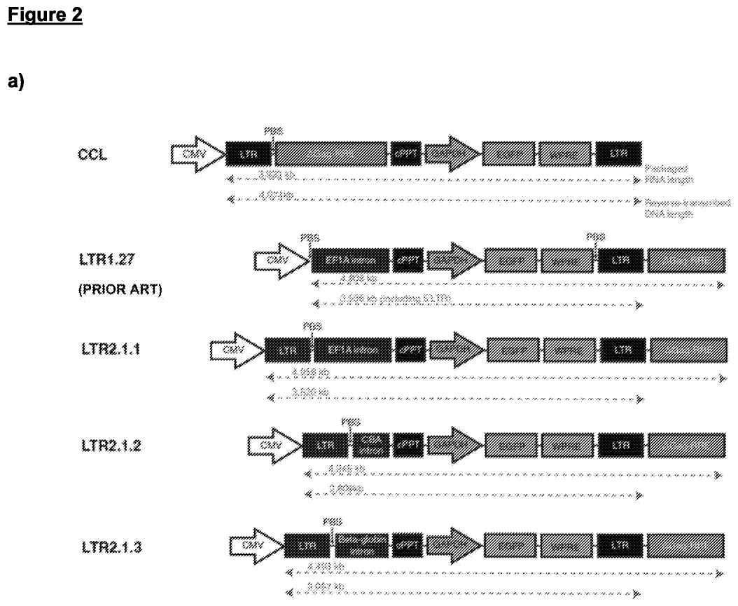

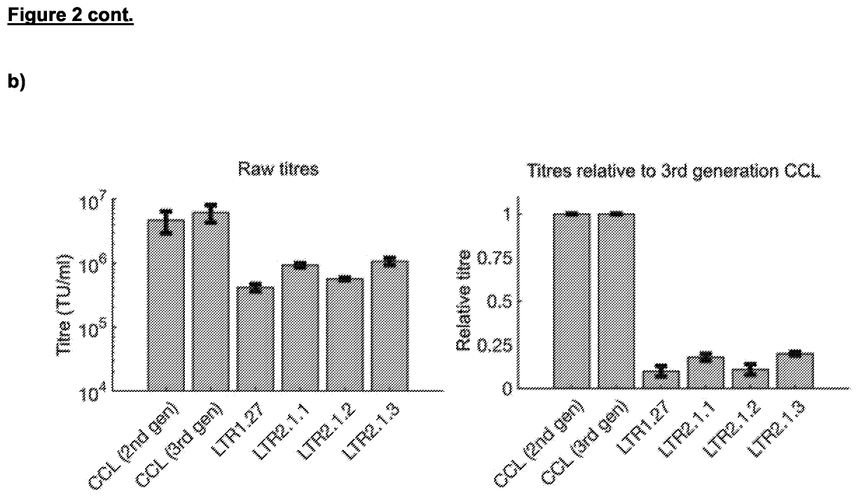

FIG. 2 are a number of schematic diagrams and graphs showing how the novel LTR1 vector designs were generated with the HIV-15′ LTR reintroduced. (a) is a vector schematic diagram showing the elements included, complete with the length of the genomic RNA (including gag-RRE) and the expected size of the DNA provirus (excluding gag-RRE). (b) are graphs where the left panel results show the EGFP titres (TU/ml) generated with each vector design and the right panel shows each titre relative to the CCL comparator (n=3 replicates per group).

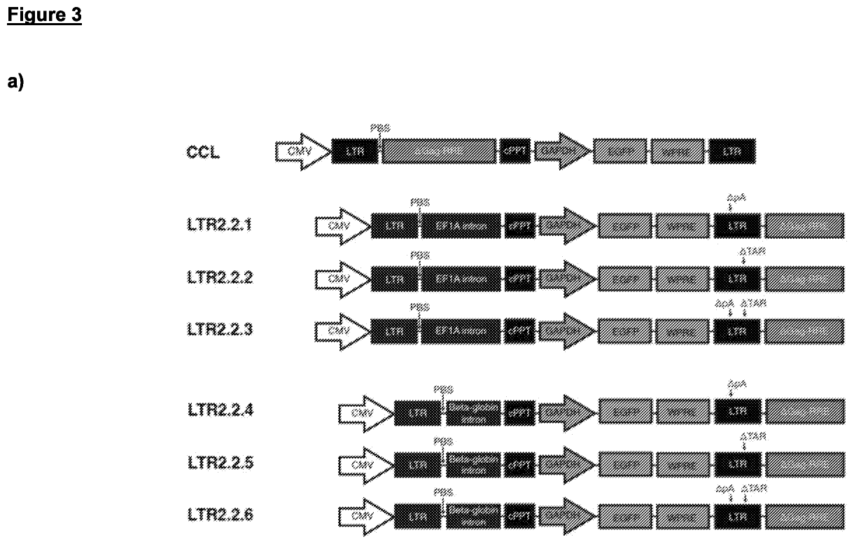

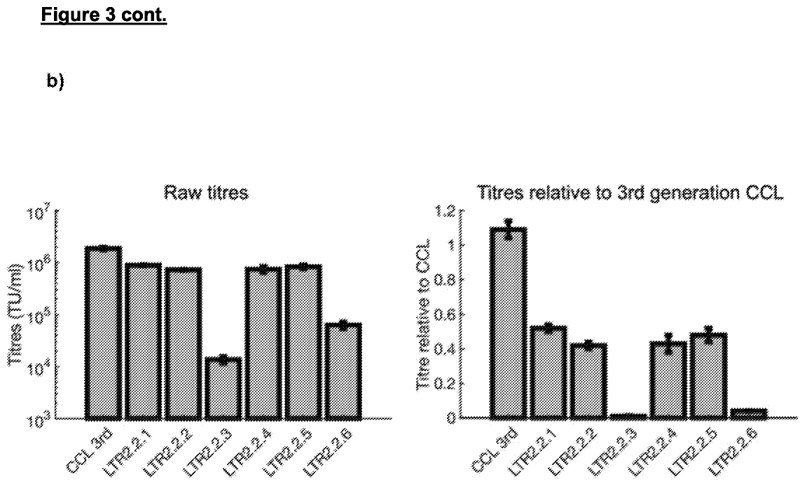

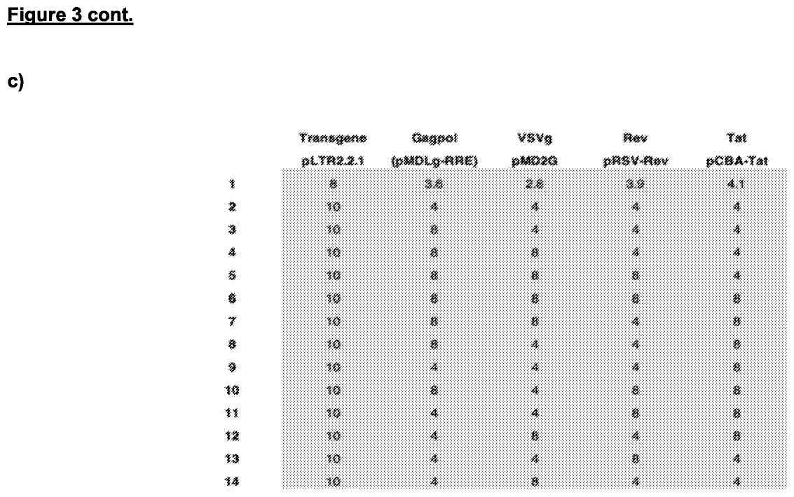

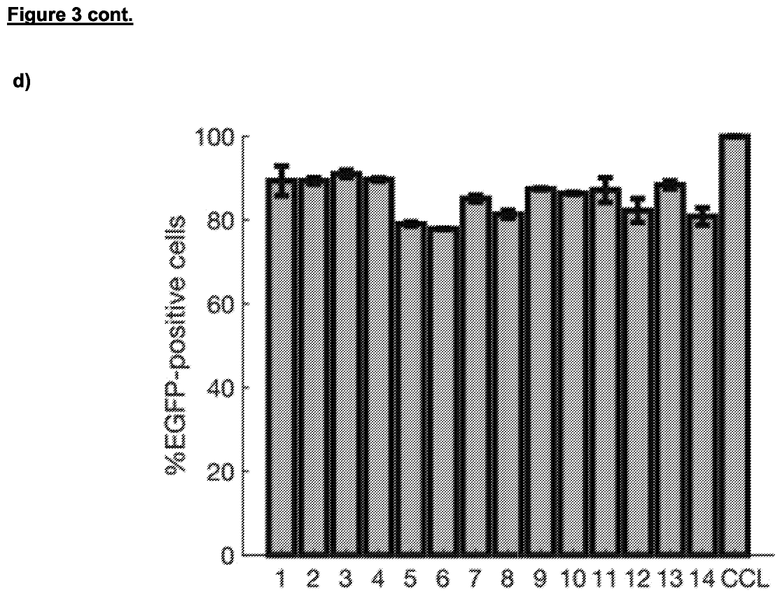

FIG. 3 are a number of schematic diagrams, graphs and a table showing the modified 3′ LTR constructs to enhance vector RNA stability. (a) is a schematic diagram showing how the novel vector designs were generated with mutations in the 3′ LTR polyA and TAR domains. (b) are graphs where the left panel results show raw titres gained from each vector and the right panel shows the titres relative to those gained with the original CCL (n=4 replicates per group). (c) is a table showing the different transfection ratios investigated (the relative amounts of each plasmid are shown in nanomolar quantities). (d) is a graph showing that the HEK 293T cells were transduced with 250 microlitres of vector supernatant and the percentage of EGFP-positive cells quantified by flow cytometry (n=2 replicates per treatment) (all transfection ratios were compared to a CCL vector prepared using the common 4:2:1:1 ratio).

FIG. 4 are a number of graphs showing the results of the testing the LTR2.1.1 vector packaging of therapeutic transgenes. Either ABCB11 or GLDC cDNA was cloned into the relevant plasmids and used to produce viral vectors. The left panel shows the titre (TU/ml) of CCL-ABCB11 and LTR2.1.1-ABCB11. The right panel shows the titre (TU/ml) of CCL-GLDC and CCL-EGFP.

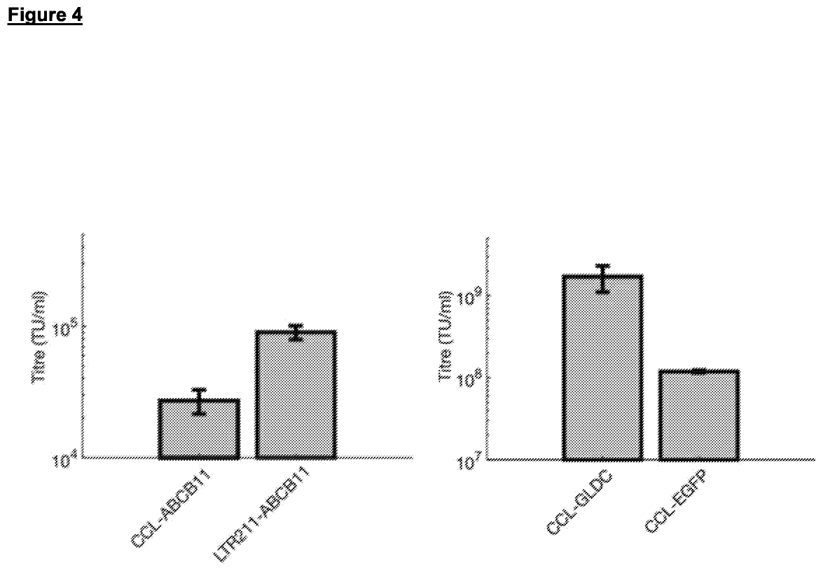

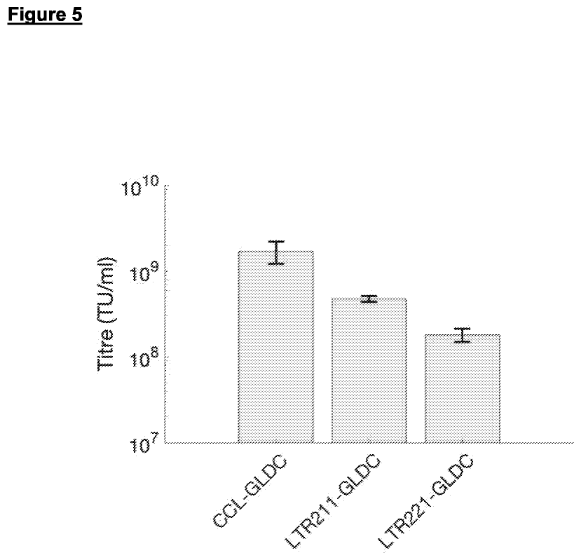

FIG. 5 is a graph showing the titre (TU/ml) of CCL-GLDC, LTR2.1.1 GLDC and LTR2.2.1 GLDC.

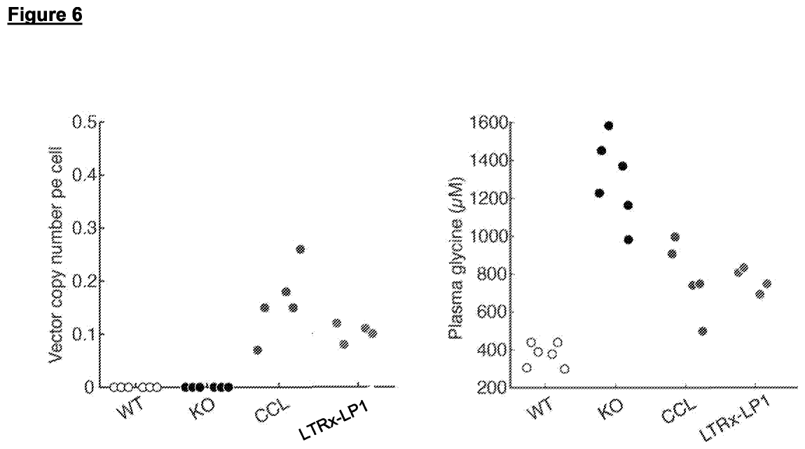

FIG. 6 are a number of graphs showing the plasma glycine values in 5-6-week-old GLDC knock-out mice after receiving neonatal gene therapy with either CCL at a dose of 5×10¹⁰ TU/kg or LTRx (promoter LP1), at a dose of 1×10¹⁰ TU/kg.

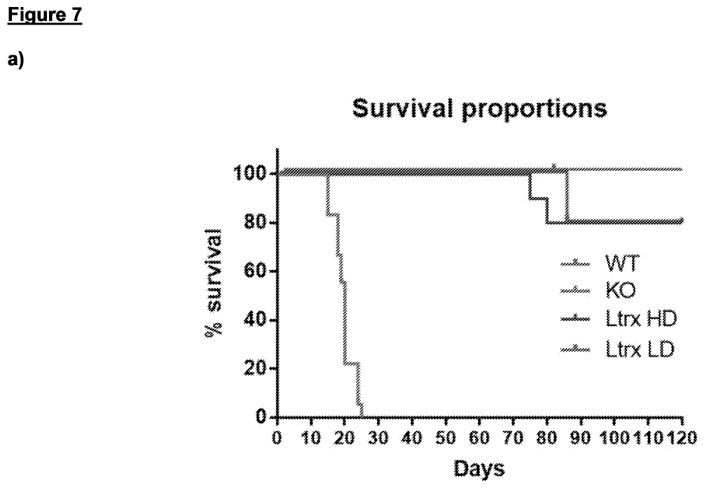

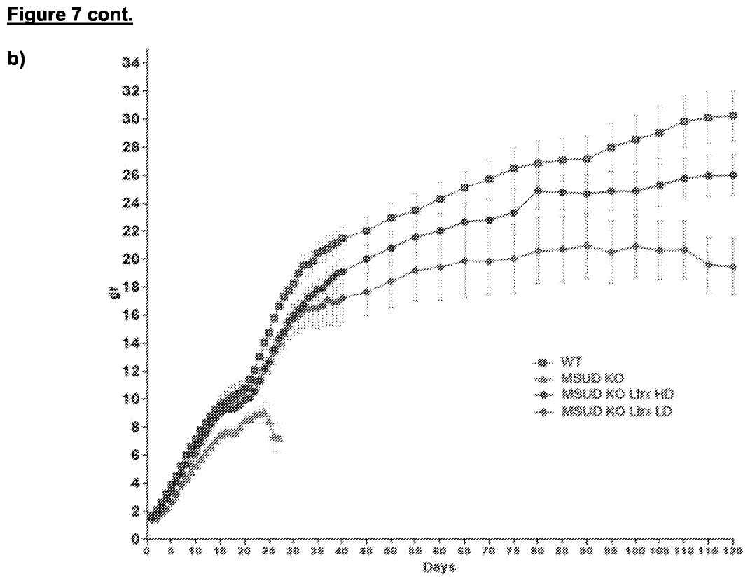

FIG. 7 are a number of graphs showing the efficacy of LTRx-Dbt treatment in a MSUD mouse model. (a) is a graph showing the survival of WT mice, MSUD KO mice and MSUD KO mice treated with either LTRx-HD or LTRx-LD. (b) is a graph showing the change in body weight in WT, MSUD KO mice and MSUD KO mice treated with either LTRx-HD or LTRx-LD. (WT: n=15; KO: n=10; LTRx-HD: n=8; LTRx-LD: n=5.) WT: Wild Type Control, MSUD KO: MSUD mouse model; HD: High dose, LD: Low dose.

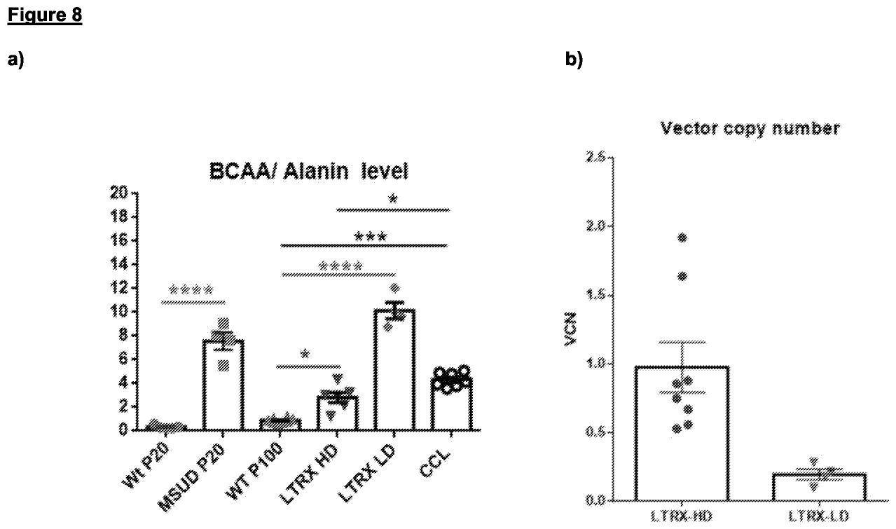

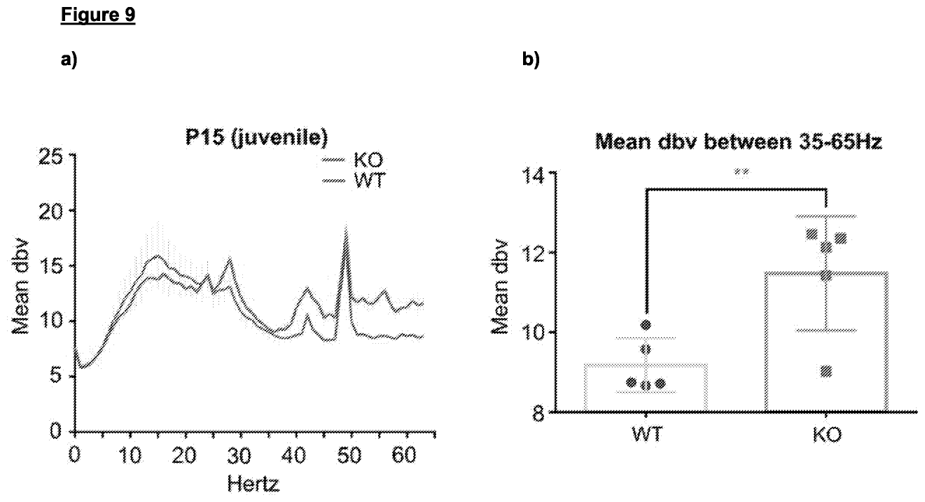

FIG. 8 are a number of graphs further showing the efficacy of LTRx-Dbt treatment in a MSUD mouse model. (a) is a graph showing the BCAA/Alanine ratio in WT and MSUD KO mice at postnatal day 20 (P20), and in WT mice, LTRx-HD, LTRx-LD and CCL-Dbt treated mice at postnatal day 100 (P100). (d) is a graph comparing the vector copy number in livers of mice treated with LTRx-HD and mice treated with LTRx-LD. One-way ANOVA with post-hoc Bonferroni correction, error bars indicate ±SEM. * p<0.05, ** p<0.01, *** p<0.001, and **** p<0.0001. WT: Wild Type; MSUD: MSUD mouse model; HD: High Dose; LD: Low dose.

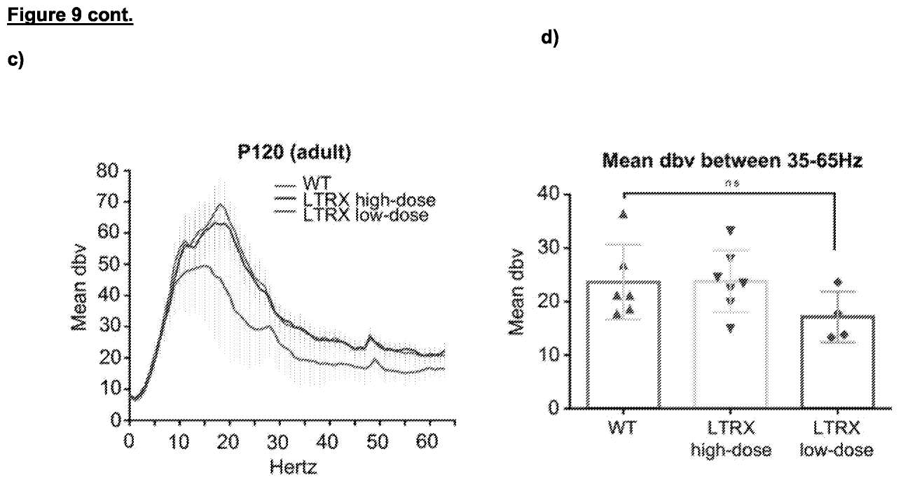

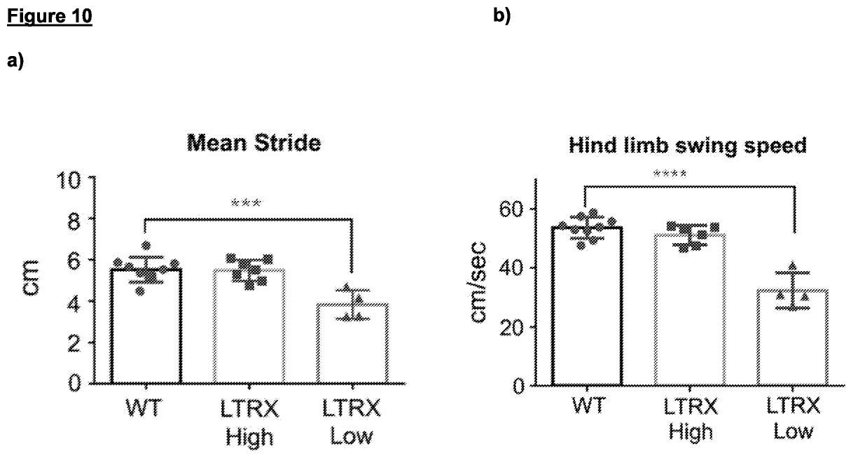

FIG. 9 are a number of graphs showing the quantification of tremor analysis at different frequencies in a MSUD KO mouse at two weeks (P15) and four months (P120) of age. (a) is a graph showing the tremor analysis between 0 and 65 Hz in a P15 (juvenile) mouse. (b) is a graph showing the quantification of the tremor analysis at a high frequency (35-65 Hz) in WT and MSUD KO mice. (c) is a graph showing the tremor analysis in P120 (adult) WT mice and mice treated with either LTRx-HD or LTRx-LD. (d) is a graph showing the quantification of the tremor analysis at a high frequency (35-65 Hz) in WT mice and mice treated with either LTRx-HD or LTRx-LD. One-way ANOVA with post hoc Bonferroni correction. Error bars indicate ±SEM. All significance shown is to wild type control group, ** p<0.01.

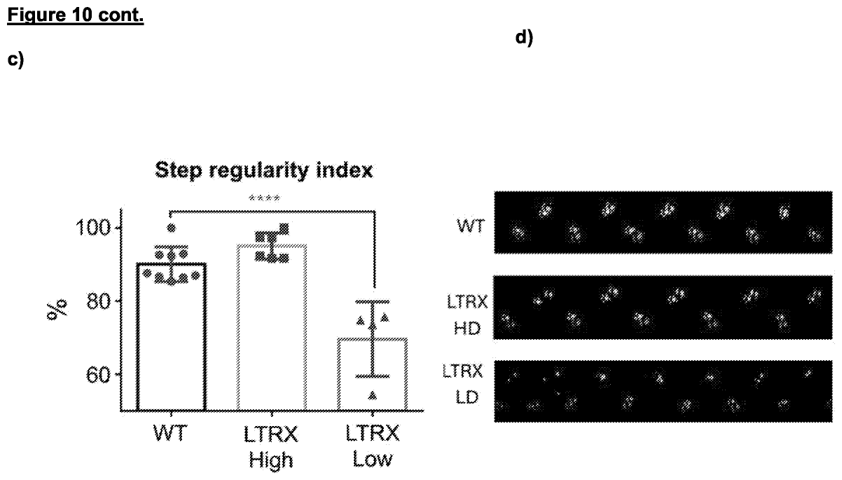

FIG. 10 are a number of graphs showing the gait analysis of MSUD mice treated with either LTRx-HD or LTRx-LD. (a) is a graph showing the quantification of mean stride (cm) of WT mice and MSUD mice treated with either LTRx-HD or LTRx-LD. (b) is a graph showing the Hind Limb Swing speed (cm/sec) of WT mice and MSUD mice treated with either LTRx-HD or LTRx-LD. (c) is a graph showing the step regularity index (%) WT mice and MSUD mice treated with either LTRx-HD or LTRx-LD. (d) is a graphical representation of paw prints captured during an average run on the CatWalk XT semi-automated gait analysis by mice at 12 weeks of age treated at P1 (Yellow: Left-Front, Cyan: Right-Front, Pink: Right-Hind and Green: Left-Hind paws). One-way ANOVA with post hoc Bonferroni correction. Error bars indicate ±SEM. All significance shown is to WT control group, ** p<0.01, *** p<0.001, and **** p<0.0001. WT: Wild Type Control, HD: High dose, LD: Low dose.

### EXAMPLES

A series of experiments were conducted in order to prepare lentiviral vectors having improved titres which could subsequently be used in gene therapy.

### Example 1—Preparing and Assessing a LTRx Vector in a NKH Mouse Model

### Materials and Methods

### Generation of Plasmid Constructs

All plasmid constructs were made using standard molecular cloning procedures and PCR-mediated deletion of plasmid sequences (29). In cases where novel elements or junctions were incorporated, synthetic DNA fragments were designed and ordered as gBlocks (Integrated DNA Technologies).

### Production of Lentiviral Vectors

LV vectors were produced as described previously (4). Briefly, 1.8×10⁷ HEK293T cells were plated per 15 cm sterile culture dish and transfected with the following components: 40 μg of the relevant transfer plasmid, 20 μg of pMDLg.RRE, 10 μg of pRSV-Rev and 10 μg of pMDG.2 (all plasmids produced by PlasmidFactory). Additionally, 10 μg of a HIV-1 Tat expression vector was supplemented for LTR vector production. The plasmid mixtures were added to 5 mL Opti-MEM and filtered through 220 nm sterile filter units. Filtered DNA was combined with 5 mL Opti-MEM (Life Tech/GE) containing 2 μM polyethylenamine (PEI, Sigma). The resulting 10 mL mixture was incubated at room temperature for 10 minutes before addition to HEK 293T cells. After 4 hours, transfection mixture was replaced with fresh culture medium. Virus supernatant was collected at 48 hours post-transfection. After each harvest, the collected medium was filtered through a cellulose acetate membrane (0.45 mm pore). In cases where vectors were concentrated, virus supernatants were placed in polyallomer centrifuge tubes (Beckman Coulter) and centrifuged for 2 hr at 90,000 xg at 4° C. in a Sorvall Discovery 90SE Centrifuge. Following centrifugation, the supernatant was removed, and pellet recovered in 200 μL phosphate-buffered saline (PBS).

### Titration of Lentiviral Vectors

Vector titration by flow cytometry: 1×10⁵ HEK293T cells were plated into each well of a 6-well plate and transduced with a dose-escalation of vector supernatant. EGFP measurements were made at 72 hours post-transduction by flow cytometry. Titres were calculated based on cell populations in the range of 5-30% EGFP+, as described previously (4).

Detection of eGFP Expression in Transduced Cells

Unless stated otherwise, 100,000 cells were analyzed for EGFP expression in a BD FACSArray Bioanalyzer. During analysis, live cells were determined by gating forward-light-scatter versus side-scatter and isolating the relevant population. EGFP-positive cells were determined by plotting EGFP fluorescence (detected using a 530/30 nm bandpass filter) versus emission from the yellow channel (detected using a 575/26 band-pass filter) to compensate for auto-fluorescence. Non-transduced controls were used to gate background expression in each channel. All flow cytometry data were analyzed by FlowJo software version 9.3.1 (Tree Star).

### In Vivo Gene Therapy in NKH Mouse Model

Homozygous NKH mice (Gldc^{GT/GT}) were crossed with Gldc^{GT/+} heterozygotes to generate knockout pups, with genotypes confirmed at postnatal day 1 (P1). Vectors were delivered to mice at P1, at a dose of 1×10¹⁰ transducing units per kilogram (TU/kg), via the superficial temporal vein. 6-weeks after vector administration, blood was sampled for metabolic analysis by mass spectrometry to quantify systemic glycine levels. Liver and various tissues were sampled for quantification of vector copy numbers (VCN) and transgene mRNA by qPCR.

### Statistical Analysis

All statistical analyses were carried out using Matlab 2015a. Student's t-test was used to compare titres and EGFP values. Titration comparisons were performed in 3 experimental replicates, unless otherwise stated.

### Restoring 5′ LTR to Strengthen Transcription Initiation

In previous experiments, LTR1 vectors were developed based on previous generation lentiviral plasmid pCCL-GADPH-EGFP and pRRL-PGK-EGRP as illustrated in FIG. 1. In these constructs, the RNA packaging sequences were transferred downstream of the 3′ self-inactivating LTR. The 5′ LTR was restored in one construct, termed LTR1.11.1, and compared to a similar construct but which lacked a 5′ LTR (LTR1.13.0). However, no significant difference in titre was found between LTR1.11.1 and LTR1.13.0. The construct that gave the highest yield relative to the third-generation vectors was LTR1.27, which lacked a 5′ LTR and contained the elongation factor 1a intron at the 5′ end of the vector.

Further optimisation was performed using the LTR1.27 vector, where the 5′ LTR was now restored adjacent to the heterologous intron. This new LTR1 prototype was termed LTR2.1. Three versions of LTR2.1 were generated, varying in the intron immediately downstream of the 5′ LTR as illustrated in FIG. 2a. These new configurations were packaged into vectors containing an enhanced green fluorescent protein (EGFP) transgene and titered. LTR2 vectors were compared versus a CCL vector and the existing LTR1.27 version to benchmark any improvements. Titration by flow cytometry showed that versions LTR2.1.1 and LTR2.1.3 were the optimal prototypes, with each of these designs providing a 2-fold titre increase over LTR1.27 (P=0.04 and 0.01 respectively) as shown in FIG. 2b. Both LTR2.1.1, which contained the elongation factor 1a intron and LTR2.1.3, which contained the B-globin intron, were brought forward to further iterative optimisation steps.

### Mutating 3′ LTR Motifs to Promote Transcriptional Read-Through

Six novel vector designs were generated from LTR2.1 and LTR2.3 as illustrated in FIG. 3a. The novel structures contained mutation of two critical regions of the 3′ LTR. The HIV-1 polyA site, located in the ‘R’ domain of the 3′ LTR, was mutated to eliminate its activity (10). Polyadenylation at this site could be problematic for LTR1, as the packaging sequences necessary for encapsidation are located downstream of this region as shown in FIG. 1.

Additionally, the HIV-1 transacting responsive (TAR) hairpin was mutated to block Tat binding to the 3′ LTR (11). This would theoretically reduce Tat-mediated transcription initiation from the 3′ LTR, which would undesirably exclude the transgene expression cassette.

Titration by flow cytometry showed that versions containing polyA mutations gave titres averaging 52% and 43% relative to CCL as shown in FIG. 3b. Vectors containing TAR mutations gave titres ranging from 42% to 48% relative to CCL. However, a combination of Tat and TAR mutations gave comparatively low titres, averaging 1% and 4% relative to CCL. Given that the titre gained from LTR2.2.1 gave the highest vector titre so far, this iteration was carried forwards for further downstream optimisation.

Production of lentiviral vectors involves co-transfection of the transgene plasmid along with three further plasmids encoding HIV-1 gagpol, the envelope protein (VSVg) and HIV-1 Rev (13). LTR1 vectors require additional transfection of a HIV-1 Tat plasmid (4). However, the relative amounts of each component have never previously been optimised in an LTR2 context.

Fourteen different transfection ratios were investigated to identify the optimal formulation for producing LTR2.2.1 vectors as shown in FIG. 3c. The relative amount of each plasmid was calculated by dsDNA molarity, rather than the conventional approach of quantifying by mass. This molarity-driven approach would account for differences in the size of each plasmid. Titration by flow cytometry showed that the optimal ratio of transgene: gagpol: envelope: rev: tat was 4:2:1:1:1 as illustrated in FIG. 3d.

### Packaging of Candidate Transgenes Reveals Importance of Metabolic Pathways to Lentiviral Vector Propagation

HIV-1-based LV vector gene therapy has shown clinical proof of concept for ex vivo haemopoietic stem cell (HSC) therapy (11-13) and in vivo brain gene therapy for Parkinson's Disease (17). Beyond these clinical applications, LV gene therapy is being developed for in vivo liver gene therapy targeting Haemophilia B, with preclinical safety having been demonstrated in dogs and non-human primates (NHPs) (18, 19).

LV has particular promise for treating paediatric liver diseases, given that integrated vector genomes will survive hepatocellular proliferation during childhood, which otherwise depletes adeno-associated virus (AAV) vectors. Experiments were therefore conducted to produce LTR2 vectors containing transgenes that could be applied to gene therapy for paediatric liver diseases. For proof of principle, Nonketotic Hyperglycinaemia (NKH), an inborn error of glycine metabolism, as well as gene therapy for Bile Salt Export Pump deficiency (BSEP), an inherited cholestasis disorder, were targeted for gene therapy.

In each case, the relevant transgene was packaged into either LTR2.1.1, LTR2.2.1, or CCL vectors. This included Glycine Decarboxylase (GLDC) for NKH and ATP binding cassette subfamily B member 11 (ABCB11) for BSEP deficiency. Surprisingly and unexpectedly, each transgene was packaged with varying efficiency in each vector backbone, despite comprising similar transgene sizes (~3 kb each).

When packaging the ABCB11 transgene, LTR2.2.1 gave >3-fold higher vector titres than CCL (P<0.001) as illustrated in FIG. 4, despite routinely producing 2-fold lower titres than CCL when packaging an EGFP transgene, shown in FIG. 3. This is potentially due to the role of bile acid metabolism in cholesterol homeostasis, which is important for maintenance of lipid rafts in the viral envelope (17). ABCB transporters have previously shown effects on lentiviral vector titres in the context of ABCB4, as overexpression of this gene in LV vector producer cells disrupted cholesterol incorporation into vector envelopes and reduced their infectivity (21).

Conversely, when attempting to package GLDC, backbone LTR2.1.1 gave a higher titre than LTR2.2.1 (P<0.001), suggesting that efficient translation of GLDC was beneficial for high titres as shown in FIG. 5, despite LTR2.2.1 usually producing greater levels of vector titre, see FIGS. 2 and 3. It is believed that LTR2.1.1 provides a higher titre when packaging GLDC transgene due to the functionality of the GLDC enzyme product in producer cells. The key difference between LTR2.1.1 and LTR2.2.1 is that the latter contains a polyA mutation in its 3′ LTR, meaning that transgene expression is suppressed in producer cells. Therefore, using LTR2.2.1-GLDC is likely to be expressed at much lower levels during vector propagation. Thus, the higher titre of LTR2.1.1-GLDC may be due to GLDC enzyme activity itself creating a permissive environment, from which LTR2.2.1 does not benefit. Additionally, CCL-GLDC titres compared to CCL-EGFP titres appears to show higher levels contained when packaging GLDC as shown in FIG. 4.

### In Vivo Data Comparing the LTR2.1.1-GLDC and the CCL-GLDC Vectors

The LTR2.1.1-GLDC construct was tested in vivo in NKH mouse models and compared against the CCL-GLDC construct shown in FIG. 6. Surprisingly, it was found that the LTR2.1.1 construct could at least match the efficacy of CCL, even when delivered at 5-fold lower dose. Statistical comparison showed no significant difference in vector copy number (P=0.3) however the LTR2.1.1-GLDC vectors were shown to reduce plasma glycine to a greater extent (P=0.05).

### CONCLUSIONS

HIV-1-based LV vectors have been developed for a variety of gene therapy applications. Generally, LV is preferred over other gene delivery platforms when the treated cells are expected to undergo successive rounds of cell division, as they permanently integrate their genetic material into the cell genome. However, LV integration has the disadvantage of potentially causing insertional mutagenesis. LTR1, a novel lentiviral vector, was developed as a potential solution to this problem, but previous LTR1 versions have been restricted by low production titres. The primary aim of this study was to engineer LTR1 technology to increase vector titres.

Iterative optimisation successfully increased vector titres by 5-fold over LTR1.27. This was achieved by engineering the vector backbone to enhance transcription initiation and minimise premature termination during vector production. This included, unexpectedly, restoration of the HIV-15′ LTR adjacent to a heterologous intron and mutation of the HIV-1 3′ LTR polyA site.

LV-mediated liver gene therapy has the potential to address unmet need in management of patients with severe early onset metabolic diseases. Proof of concept for LV gene therapy for inborn errors of metabolism (IEMs) has been demonstrated in models of hereditary tyrosinaemia, glycogen storage disease and methylmalonic acidaemia (23-25). LV is potentially advantageous over AAV in this area given its long-term gene expression profile in dividing hepatocytes. A further advantage over AAV is that LV vectors are coated with a glycoprotein derived from Vesicular Stomatitis Virus (VSVg), which primarily infects livestock, thus seropositivity in the human population is extremely low (26). Particle doses required for efficacy have been lower for LV (1×10¹⁰ vg/kg for LV, versus 1×10¹⁴ vg/kg for AAV (27), which potentially mitigates adverse events associated with capsid-induced hepatotoxicity in AAV clinical trials (27).

The LTR2.2.1 construct differs from LTR2.1.1 by a polyA mutation in the 3′ LTR, which was originally inserted with the aim of allowing transcriptional read-through into downstream packaging sequences, thereby maximising the number of packageable genomes. However, as a side-effect, this mutation also reduced transgene expression in producer cells, potentially due to subgenomic transgene mRNAs lacking polyadenylation. Despite this, this difference was not observed in treated cells, potentially due to the complete 5′ LTR (containing functional polyA motif) being copied to the 3′ LTR during reverse-transcription.

This aspect of the vectors made a surprising benefit to titres when attempting to package candidate therapeutic transgenes into LTR2.1.1 and LTR2.2.1 for proof of principal gene therapy studies. It is known from previous studies that overexpression of transgenes involved in cholesterol homeostasis (e.g., ABCB4) during LV vector production destabilises viral envelopes, causing significant titre reduction, due to dysregulation of cholesterol transport across the cell membrane (21). It has been proposed that ABCB11 expression also affects cholesterol homeostasis, as its overexpression in mice causes hypercholesterolaemia due to enhanced cholesterol absorption into treated cells (28). When packaging an ABCB11 transgene into LTR2.2.1 vectors, higher vector titres were observed compared to those achieved with a conventional CCL vector, likely due to suppression of ABCB11 translation as a result of the polyA mutation. This method of blocking ABCB11 expression during vector production may normalise cholesterol homeostasis and therefore may preserve vector titre efficiency.

Conversely, the opposite trend was observed when attempting to package a GLDC transgene, as LTR2.2.1 gave a lower titre than LTR2.1.1. It was concluded that this was due to a positive effect of GLDC on vector propagation, which was lost when using the LTR2.2.1 backbone, due to lack of transgene polyadenylation. The precise mechanism may be linked to the role of this enzyme in nucleotide metabolism, where high GLDC expression has been proposed to promote viral replication (22).

In vivo studies revealed a further benefit of the LTR2.1.1 vector construct. Lower particle doses of LTR2.1.1-GLDC were required to achieve the same efficacy as CCL-GLDC in NKH mouse models. This translates to a greater clinical benefit as lower particle doses are proposed to reduce the number of integration events and activation of a strong immune response in a patient.

### Example 2—Validation of the LTRx Vector in a MSUD Mouse Model

### Materials and Methods

### Production of LTRx-Dbt Vectors

LV vectors were produced as described previously (4). Briefly, 2×10⁷ HEK293T cells were plated per T175 culture flasks with the following components: 40 μg of the transgene plasmid (Ltr2.1.1-LPS-Dbt), 20 μg of pMDLg.RRE, and 10 μg of pMDG.2 (all plasmids produced by PlasmidFactory) and 10 μg of pRSV-Rev-pCMV-Tat. The plasmid mixtures were added to 5 mL Opti-MEM and filtered through 220 nm sterile filter units. Filtered DNA was combined with 5 mL Opti-MEM (Life Tech/GE) containing 1:1 ratio of μl of PEIpro® transfection reagent (Polyplus) to μg of DNA. The resulting 10 mL mixture was incubated at room temperature for 10 min before addition to HEK293T cells. Virus supernatant was collected at 48 h post-transfection. After harvest, the collected medium was filtered through a cellulose acetate membrane (0.45 mm pore). Briefly, viruses were placed in 50 ml falcon tubes and centrifuged over night at 14,000 rpm×g at 4° C. in an Eppendorf 5810R Centrifuge. Following centrifugation, the supernatant was removed, and pellet recovered in 200 μL PBS.

### Titration of LTRx-Dbt Vectors

1×10⁵ HEK293T cells were plated into each well of a 6-well plate and transduced with a dose escalation of concentrated LV. Cells were passaged into 1:3 ratio after a week and kept in the culture for 5 days. Cells were then harvested and DNA was extracted using Qiagen DNeasy Blood & Tissue Kit following manufacturer protocol. Taqman (Applied Biosystems™ TaqMan™ Universal Master Mix) qPCR assay was used to identify the LV titre. The qPCR was run for each sample in duplicates, and analysis was based on the average of the duplicate Cq values. Primer and TaqMan probes were designed against beta-actin and LTRx backbone. Probes were labelled with a FAM reporter fluorophore and a quencher at the 5′ and 3′ end. Primers and probes were produced at Sigma-Aldrich (St. Luis, USA). Thermocycling conditions were 2 min at 50° C., followed by initial denaturation at 95° C. for 2 min and 45 cycles at 95° C. for 15 s (denaturation) and 60 s at 56° C. (annealing and extension) in a qPCR machine (4376357, Thermo Fisher, Dartford, UK). Template standard plasmid containing the partial Beta-actin and Ltrx backbone sequence with a known concentration (10⁷, 10⁶, 10⁵, 10⁴, 10³ number of molecules/μl) was used to achieve standard curve and calculating the vector copy number and titre based on the Cq values.

### Vector Copy Number Analysis

Liver DNA was extracted using Qiagen DNeasy Blood & Tissue Kit following manufacturer protocol. Extracted DNA was quantified by qPCR Power SYBR™ Green PCR Master Mix (Applied Biosystems 4368577). The qPCR was run for each sample in duplicates, and analysis was based on the average of the duplicate Cq values. Each sample was analysed for vector content using primers for Dbt and for content of the house keeping gene (mouse titin gene). Standards for LTRx and the housekeeping gene content respectively consisted of in-house cloned plasmids containing the relevant section of the titin sequence. The known concentrations of the of the standard plasmid (10⁷, 10⁶, 10⁵, 10⁴, 10³ number of molecules/μl) was used to calculate the vector copy number in the liver. Thermocycling conditions were 2 min at 50° C., followed by initial denaturation at 95° C. for 10 min and 40 cycles at 95° C. for 15 s (denaturation) and 60 s at 60° C. (annealing and extension) in a qPCR machine (4376357, Thermo Fisher, Dartford, UK).

### The MSUD Mouse Model

All animal studies were approved by the UK Home Office for the conduct of regulated procedures under license (Animal Scientific Procedures Act, 1986). Dbt^{m1GehTg} (Cebpb-(TA) 5Bjd Tg (tetO-DBT) A1Geh/J line (JAX stock #006999, (31)) was used in this study. Crossing the heterozygous mouse resulted in mDbt-hDbt+, mDbtthDbt+animals, which had been used as a MSUD KO and WT Control in this study. mDbt-hDbt-animals were excluded from the study due to severity of the phenotype which causes early postnatal lethality. Animals were kept under a 12 h light/dark cycle and provided normal food and water ad libitum. For survival, any mice that lost 15% of their body weight after a 24 h period or showed ataxia/movement difficulties were considered to have reached their humane endpoint and sacrificed. Mice underwent terminal exsanguination by trans-cardiac perfusion with phosphate-buffered saline. Liver and other organs were subsequently extracted and snap-frozen on dry ice and stored at −80° C. for downstream analysis.

### Neonatal Administration of LV Vector:

The P1 time point was selected for gene delivery. 4.5 E10 TU/kg of the LTRx-Dbt vector (High Dose, HD) or 4×times dilution (Low Dose, LD) was administrated intravenously injections via the superficial temporal vein using a 33-gauge needle (Hamilton, Reno, NV, USA). The mice were allowed to recover and return to the cage with the mother.

### Behavioural Analysis

### Tremor

Tremor analysis was carried out on wild type control, MSUD KO and treated MSUD LTRX-Dbt mice at 2 weeks (P15) or four months (P120) of age using a commercial tremor monitor (San Diego Instruments, San Diego, CA, USA) as previously described (33). Individual mice were placed inside the apparatus on an anti-vibration table and monitored for 256 s, after 30s of acclimatisation time. The output (amplitude/time) was analysed using LabView software, to give a measurement of power at each monitored frequency (0-64 Hz).

### Gait

Gait monitoring was performed on control, untreated, and wild type control and treated MSUD LTRX-Dbt mice at 4 months using the automated gait analysis CatWalk system (Noldus, Wageningen, The Netherlands). The mice were monitored by being individually placed at one end of the CatWalk and were filmed freely walking across a filmed section with a backlit stage.

A minimum of three successful runs were recorded per session, where a run across the stage was deemed successful if standard run criteria were met. Paw prints during successful runs were checked, classified, and analysed using the CatWalk XT software v10.6 (Noldus) to produce overall run measurements.

### Determination of BCAA Serum Concentration

To obtain blood serum, animals were anaesthetized with isoflurane in 100% oxygen, and blood was obtained via heart puncture using 1 ml Insulin Syringes. The blood was immediately transferred to MiniCollect® tubes (0.5 ml LH Lithium Heparin) and centrifuged at 4000×g for 20 minutes. Plasma was then removed and snap-frozen on dry ice. BCAA levels of serum samples were obtained using the branched-chain amino acid assay kit from Abcam (#ab83374) according to the manufacturer's specifications. BCAA levels typically increase after feeding. To account for this, the concentration of L-Alanine was measured using the L-Alanine Assay Kit (#ab83394). The normalized level of BCAA/Alanine was used as an indicator of BCAA increase in MSUD animals.

### Statistical Analysis

Statistical analysis tailored to each experiment was performed using GraphPad Prism version 9. To assess for statistical differences in dual comparisons two-tailed Student's t-test was applied, whereas for multiple comparisons one-way or two-way ANOVA was performed with Bonferroni post hoc correction test.

### Assessment of the Efficacy of LTRx-Dbt Treatment in a MSUD Mouse Model

Maple Syrup Urine Disease (MSUD) is a metabolic disorder caused by a deficiency in branched-chain keto acid dehydrogenase (BCKDH) resulting in elevated levels of branched-chain amino acids (BCAA) in the blood. Patients can suffer from developmental delays, feeding difficulties, neurotransmitter imbalances, and encephalopathic syndrome. BCKDH is expressed in various tissues, but the liver is the primary organ responsible for BCAA metabolism. BCKDH is a complex enzyme comprising three essential subunits: a-ketoacid dehydrogenase, dihydrolipoyl transacylase (DBT), and dihydrolipoyl dehydrogenase. Deficiency in any of these subunits results in the disease manifestation of MSUD. Specifically, mutations in the DBT gene are presenting varying symptoms depending on the residual enzyme activity (30).

To further validate LTRx platform, the efficacy of LTRx based lentiviral vectors expressing the Dbt gene and containing a liver specific promoter (LPS) were investigated in a MSUD mouse model. In this mouse model, the mouse Dbt gene has been knocked out and replaced by the human Dbt gene, leading to a partial restoration of BCKDH enzymatic activity. Consequently, the animals display symptoms typical of the intermediate form of MSUD (31). The treated mice were injected with either a high dose (HD) or low dose (LD) of the LTRx-Dbt vector and were assessed for survival, weight, and BCAA during a 4-month period.

As shown in FIGS. 7A and 7B, neonatal injection of LTRx-Dbt vector significantly increased the life span of MSUD KO mice compared to the untreated MSUD KO mice. LTRx-Dbt treated mice further showed an improvement in weight gain compared to the untreated mice. The BCAA/Alanine plasma level was also dramatically improved and reduced from the 23-fold increase over aged-matched wild-type control in MSUD KO mice to only three-fold after treatment with high-dose LTRx-Dbt. There was a significant decrease in BCAA levels when comparing LTRx-HD animals to the experimental cohort treated with CCL-Dbt (3rd generation lentiviral vector) despite receiving 2.5 times higher dose of vector injection, as shown in FIG. 8A.

To further assess the efficacy of the injection the vector copy number (VCN) per cell in the liver was measured. The LTRx-HD group showed about 4× higher VCN compared to the LTRx-LD cohort as illustrated in FIG. 8B.

### Assessing the Effect of LTRx—Dbt Administration on Ataxia and Locomotor Function in a MSUD Mouse Model

Patients with MSUD can suffer from ataxia which is caused by catabolic distress (32). The experimental cohorts were assessed for ataxia and locomotor function using an automated tremor sensor and Catwalk-XT gait analysis. The gait parameters investigated were the Mean Stride, which refers to the distance (cm) between successive paw placements, the Hind Limb Swing speed (cm/sec), which refers to the speed of movement of hind paws between successive placements, and the Step Regularity Index, which refers to the percentage of normal step sequence patterns during an uninterrupted run, thus measuring the degree of interlimb coordination.

Tremor analysis revealed that the untreated MSUD KO mouse showed the characteristic tremor compared to the wild-type control at higher frequency, as illustrated in FIGS. 9A and B; this was ameliorated in the animals treated with LTRX-Dbt as shown in FIGS. 9C and D.

An examination of the gait traces revealed a significant deterioration in MSUD animals treated with low-dose LTRx-Dbt vector. The quantification of stride length, step regularity index and hind limb swing speed showed marked improvement in MSUD mice given high-dose treatment, suggesting the existence of a therapeutic threshold for reducing BCAAs as shown in FIG. 10.

The experiments conducted show two new lentiviral vector structures for mediating gene therapy. Each design could be selected for specific transgenes, depending on the behaviour of the transgene in cells producing HIV-1 components. Additionally, two metabolic pathways have been identified that can potentially affect lentiviral vector titres.

The foregoing embodiments are not intended to limit the scope of protection of the claims, but rather to describe examples of how the invention may be put into practice.

Sequence Listings

SEQ ID No. 1 Cytomegalovirus (CMV) promoter
gacattgattattgactagttattaatagtaatcaattacggggtcattagttcatagcccatatatggagttccgcgttacataactt
acggtaaatggcccgcctggctgaccgcccaacgacccccgcccattgacgtcaataatgacgtatgttcccatagtaacgc
caatagggactttccattgacgtcaatgggtggagtatttacggtaaactgcccacttggcagtacatcaagtgtatcatatgcc
aagtacgccccctattgacgtcaatgacggtaaatggcccgcctggcattatgcccagtacatgaccttatgggactttcctact
tggcagtacatctacgtattagtcatcgctattaccatggtgatgcggttttggcagtacatcaatgggcgtggatagcggtttga
ctcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagagctcgtttagtga
acc
SEQ ID No. 2_5′ long terminal repeat (LTR)
gggtctctctggttagaccagatctgagcctgggagctctctggctaactagggaacccactgcttaagcctcaataaagcttg
ccttgagtgcttcaagtagtgtgtgcccgtctgttgtgtgactctggtaactagagatccctcagacccttttagtcagtgtggaaa
atctctagca
SEQ ID No. 3 Primer binding site (PBS)
tggcgcccgaacagggac
Primer overextension sequence
[c/t]tgaaa
SEQ ID No. 4 Elongation factor 1α intron (EF1A)
gtaagtgccgtgtgtggttcccgcgggcctggcctctttacgggttatggcccttgcgtgccttgaattacttccacctggctgcag
tacgtgattcttgatcccgagcttcgggttggaagtgggtgggagagttcgaggccttgcgcttaaggagccccttcgcctcgtg
cttgagttgaggcctggcctgggcgctggggccgccgcgtgcgaatctggtggcaccttcgcgcctgtctcgctgctttcgata
agtctctagccatttaaaatttttgatgacctgctgcgacgctttttttctggcaagatagtcttgtaaatgcgggccaagatctgca
cactggtatttcggtttttggggccgcgggcggcgacggggcccgtgcgtcccagcgcacatgttcggcgaggcggggcctg
cgagcgcggccaccgagaatcggacgggggtagtctcaagctggccggcctgctctggtgcctggcctcgcgccgccgtgt
atcgccccgccctgggcggcaaggctggcccggtcggcaccagttgcgtgagcggaaagatggccgcttcccggccctgc
tgcagggagctcaaaatggaggacgcggcgctcgggagagcgggcgggtgagtcacccacacaaaggaaaagggcct
ttccgtcctcagccgtcgcttcatgtgactccacggagtaccgggcgccgtccaggcacctcgattagttctcgagcttttggagt
acgtcgtctttaggttggggggaggggttttatgcgatggagtttccccacactgagtgggtggagactgaagttaggccagctt
ggcacttgatgtaattctccttggaatttgccctttttgagtttggatcttggttcattctcaagcctcagacagtggttcaaagtttttttc
ttccatttcag
SEQ ID No. 5 β globin intron
gtgagtctatgggacccttgatgttttctttccccttcttttctatggttaagttcatgtcataggaaggggagaagtaacagggtac
acatattgaccaaatcagggtaattttgcatttgtaattttaaaaaatgctttcttcttttaatatacttttttgtttatcttatttctaata
ctttccctaatctctttctttcagggcaataatgatacaatgtatcatgcctctttgcaccattctaaagaataacagtgataatttctgggt
taaggcaatagcaatatttctgcatataaatatttctgcatataaattgtaactgatgtaagaggtttcatattgctaatagcagcta
caatccagctaccattctgcttttattttatggttgggataaggctggattattctgagtccaagctaggcccttttgctaatcatgttc
atacctcttatcttcctcccacag
SEQ ID No. 6 Central polypurine tract (cPPT) and central termination site (CTS)
ttttaaaagaaaaggggggattggggggtacagtgcaggggaaagaatagtagacataatagcaacagacatacaaact
aaagaattacaaaaacaaattacaaaaattcaaaatttt
SEQ ID No. 7 Woodchuck Hepatitis Virus (WPRE)
aatcaacctctggattacaaaatttgtgaaagattgactggtattcttaactatgttgctccttttacgctatgtggatacgctgcttta
atgcctttgtatcatgctattgcttcccgtatggctttcattttctcctccttgtataaatcctggttgctgtctctttatgaggagttgtggc
ccgttgtcaggcaacgtggcgtggtgtgcactgtgtttgctgacgcaacccccactggttggggcattgccaccacctgtcagc
tcctttccgggactttcgctttccccctccctattgccacggcggaactcatcgccgcctgccttgcccgctgctggacaggggct
cggctgttgggcactgacaattccgtggtgttgtcggggaagctgacgtcctttccatggctgctcgcctgtgttgccacctggatt
ctgcgcgggacgtccttctgctacgtcccttcggccctcaatccagcggaccttccttcccgcggcctgctgccggctctgcggc
ctcttccgcgtcttcgccttcgccctcagacgagtcggatctccctttgggccgcctccccgc
SEQ ID No. 8 3′ self-inactivating long terminal repeat (SIN-LTR)
tggaagggctaattcactcccaacgaagacaagatctgctttttgcttgtactgggtctctctggttagaccagatctgagcctgg
gagctctctggctaactagggaacccactgcttaagcctcaataaagcttgccttgagtgcttcaagtagtgtgtgcccgtctgtt
gtgtgactctggtaactagagatccctcagacccttttagtcagtgtggaaaatctctagca
SEQ ID No. 9 3′ long terminal repeat (LTR) with polyadenylation (polyA) site mutation
tggaagggctaattcactcccaacgaagacaagatctgctttttgcttgtactgggtctctctggttagaccagatctgagcctgg
gagctctctggctaactagggaacccactgcttaagcctcaaCaaagcttgccttgagtgcttcaagtagtgtgtgcccgtctgt
tgtgtgactctggtaactagagatccctcagacccttttagtcagtgtggaaaatctctagca
SEQ ID No. 10 3′ long terminal repeat (LTR) with HIV-1 transacting responsive (TAR)
hairpin site mutation
tggaagggctaattcactcccaacgaagacaagatctgctttttgcttgtactgggtctctctggttagaccagatctgagcctgg
gagTtctctggctaactagggaacccactgcttaagcctcaataaagcttgccttgagtgcttcaagtagtgtgtgcccgtctgtt
gtgtgactctggtaactagagatccctcagacccttttagtcagtgtggaaaatctctagca
SEQ ID No. 11 3′ long terminal repeat (LTR) with polyadenylation (polyA) site mutation
and HIV-1 transacting responsive (TAR) hairpin hairpin site
tggaagggctaattcactcccaacgaagacaagatctgctttttgcttgtactgggtctctctggttagaccagatctgagcctgg
gagTtctctggctaactagggaacccactgcttaagcctcaacaaagcttgccttgagtgcttcaagtagtgtgtgcccgtctgt
tgtgtgactctggtaactagagatccctcagacccttttagtcagtgtggaaaatctctagca
SEQ ID No. 12 HIV-1 psi packaging sequence
ctctctcgacgcaggactcggcttgctgaagcgcgcacggcaagaggcgaggggcggcgactggtgagtacgccaaaaa
ttttgactagcggaggctagaaggagagagatgggtgcgagagcgtc
SEQ ID No. 13 Rev Response Element (RRE)
aggagctttgttccttgggttcttgggagcagcaggaagcactatgggcgcagcgtcaatgacgctgacggtacaggccaga
caattattgtctggtatagtgcagcagcagaacaatttgctgagggctattgaggcgcaacagcatctgttgcaactcacagtct
ggggcatcaagcagctccaggcaagaatcctggctgtggaaagatacctaaaggatcaacagctcct
SEQ ID No. 14 ΔGag-RRE
ctctctcgacgcaggactcggcttgctgaagcgcgcacggcaagaggcgaggggcggcgactggtgagtacgccaaaaa
ttttgactagcggaggctagaaggagagagatgggtgcgagagcgtcagtattaagcgggggagaattagatcgcgatgg
gaaaaaattcggttaaggccagggggaaagaaaaaatataaattaaaacatatagtatgggcaagcagggagctagaac
gattcgcagttaatcctggcctgttagaaacatcagaaggctgtagacaaatactgggacagctacaaccatcccttcagac
aggatcagaagaacttagatcattatataatacagtagcaaccctctattgtgtgcatcaaaggatagagataaaagacacc
aaggaagctttagacaagatagaggaagagcaaaacaaaagtaagaccaccgcacagcaagcggccgctgatcttcag
acctggaggaggagatatgagggacaattggagaagtgaattatataaatataaagtagtaaaaattgaaccattaggagt
agcacccaccaaggcaaagagaagagtggtgcagagagaaaaaagagcagtgggaataggagctttgttccttgggttct
tgggagcagcaggaagcactatgggcgcagcgtcaatgacgctgacggtacaggccagacaattattgtctggtatagtgc
agcagcagaacaatttgctgagggctattgaggcgcaacagcatctgttgcaactcacagtctggggcatcaagcagctcc
aggcaagaatcctggctgtggaaagatacctaaaggatcaacagctcctggggatttggggttgctctggaaaactcatttgc
accactgctgtgccttggaatgctagttggagtaataaatctctggaacagatttggaatcacacgacctggatggagtggga
cagagaaattaacaattacacaagcttaatacactccttaattgaagaatcgcaaaaccagcaagaaaagaatgaacaag
aattattggaattagataaatgggcaagtttgtggaattggtttaacataacaaattggctgtggtatataaaattattcataatgat
agtaggaggcttggtaggtttaagaatagtttttgctgtactttctatagtgaatagagttaggcagggatattcaccattatcgtttc
agacccacctcccaaccccgaggggacccgacaggcccgaaggaatagaagaagaaggtggagagagagacagag
acagatccattcgattagtgaac

### REFERENCES

1. Cunningham S C, Dane A P, Spinoulas A, Alexander I E. Gene Delivery to the Juvenile Mouse Liver Using AAV2/8 Vectors. Mol Ther 2008; 16:1081-1088.
2. Mingozzi F, High K A. Immune responses to AAV vectors: overcoming barriers to successful gene therapy. Blood 2013; 122:23-36.
3. Cavazzana-Calvo M, Payen E, Negre O, Wang G, Hehir K, Fusil F et al. Transfusion independence and HMGA2 activation after gene therapy of human β-thalassaemia. Nature 2010; 467:318-22.
4. Vink C A, Counsell J R, Perocheau D P, Karda R, Buckley SMKK, Brugman M H et al. Eliminating HIV-1 Packaging Sequences from Lentiviral Vector Proviruses Enhances Safety and Expedites Gene Transfer for Gene Therapy. Mol Ther 2017; 9:10-20.
5. Counsell J R, Vink C A, Perocheau DPB, Karda R, Ng J, Buckley SMK et al. 529. Novel LTR-1 Lentiviral Vectors Are Fully Functional Following the Removal of HIV-1 Gag-RRE Sequences. Mol Ther 2015; 23: S212.
6. Counsell J R, Vink C A, Karda R, Perocheau D P, Brugman M H, Buckley S M et al. 78. LTR1 Vectors Enhance Safety in Gene Therapy and Can Be Exploited for Rapid, Transient Gene Delivery. Mol Ther 2017; 25:38.
7. Huelsmann P M, Hofmann A D, Knoepfel S A, Popp J, Rauch P, Di Giallonardo F et al. A suicide gene approach using the human pro-apoptotic protein tBid inhibits HIV-1 replication. BMC Biotechnol 2011; 11:4.
8. Gray S J, Foti S B, Schwartz J W, Bachaboina L, Taylor-Blake B, Coleman J et al. Optimizing promoters for recombinant adeno-associated virus-mediated gene expression in the peripheral and central nervous system using self-complementary vectors. Hum Gene Ther 2011; 22:1143-1153.
9. Beerli R R, Segal D J, Dreier B, Barbas C F. Toward controlling gene expression at will: Specific regulation of the erbB-2/HER-2 promoter by using polydactyl zinc finger proteins constructed from modular building blocks. Proc Natl Acad Sci USA 1998; 95:14628-14633.
10. Orkin S H, Cheng T C, Antonarakis S E, Kazazian H H. Thalassemia due to a mutation in the cleavage-polyadenylation signal of the human beta-globin gene. EMBO J 1985; 4:453-456.
11. Emiliani S, Van Lint C, Fischle W, Paras P, Ott M, Brady J et al. A point mutation in the HIV-1 Tat responsive element is associated with postintegration latency. Proc Natl Acad Sci USA 1996; 93:6377-81.
12. Chavez A, Scheiman J, Vora S, Pruitt B W, Tuttle M, P R lyer E et al. Highly efficient Cas9-mediated transcriptional programming. Nat Methods 2015; 12:326-328.
13. Dull T, Zufferey R, Kelly M, Mandel R J, Nguyen M, Trono D et al. A third-generation lentivirus vector with a conditional packaging system. J Viro/1998; 72:8463-8471.
14. Biffi A, Montini E, Lorioli L, Cesani M, Fumagalli F, Plati T et al. Lentiviral Hematopoietic Stem Cell Gene Therapy Benefits Metachromatic Leukodystrophy. Science (80-) 2013; 341:1233158-1233158.
15. Sessa M, Lorioli L, Fumagalli F, Acquati S, Redaelli D, Baldoli C et al. Lentiviral haemopoietic stem-cell gene therapy in early-onset metachromatic leukodystrophy: an ad-hoc analysis of a non-randomised, open-label, phase 1/2 trial. Lancet 2016; 388:476-487.
16. Ribeil J-A, Hacein-Bey-Abina S, Payen E, Magnani A, Semeraro M, Magrin E et al. Gene Therapy in a Patient with Sickle Cell Disease. N Engl J Med 2017; 376:848-855.
17. Palfi S, Gurruchaga J M, Ralph G S, Lepetit H, Lavisse S, Buttery P C et al. Long-term safety and tolerability of ProSavin, a lentiviral vector-based gene therapy for Parkinson's disease: a dose escalation, open-label, phase 1/2 trial. Lancet 2014; 383:1138-1146.
18. Cantore A, Ranzani M, Bartholomae C C, Volpin M, Valle P Della, Sanvito F et al. Liver-directed lentiviral gene therapy in a dog model of hemophilia B. Sci Transl Med 2015; 7: 277ra28.
19. Milani M, Annoni A, Moalli F, Liu T, Cesana D, Calabria A et al. Phagocytosis-shielded lentiviral vectors improve liver gene therapy in nonhuman primates. Sci Transl Med 2019; 11: eaav7325.
20. Lu K, Heng X, Summers M F. Structural determinants and mechanism of HIV-1 genome packaging. J Mol Biol 2011; 410:609-33.
21. van Til N P, Heutinck K M, van der Rijt R, Paulusma C C, van Wijland M, Markusic D M et al. Alteration of viral lipid composition by expression of the phospholipid floppase ABCB4 reduces HIV vector infectivity. Retrovirology 2008; 5:14.
22. Zhou J, Wang D, Wong B H-Y, Li C, Poon V K-M, Wen L et al. Identification and characterization of GLDC as host susceptibility gene to severe influenza. EMBO Mol Med 2019; 11: e9528.
23. Kaiser R A, Nicolas C T, Allen K L, Chilton J A, Du Z, Hickey R D et al. Hepatotoxicity and Toxicology of In Vivo Lentiviral Vector Administration in Healthy and Liver-Injury Mouse Models. Hum Gene Ther Clin Dev 2019; 30:57-66.
24. Clar J, Mutel E, Gri B, Creneguy A, Stefanutti A, Gaillard S et al. Hepatic lentiviral gene transfer prevents the long-term onset of hepatic tumours of glycogen storage disease type 1a in mice. Hum Mol Genet 2015; 24:2287-2296.
25. Wong ESY, Mcintyre C, Peters H L, Ranieri E, Anson D S, Fletcher J M. Correction of Methylmalonic Aciduria In Vivo Using a Codon-Optimized Lentiviral Vector. Hum Gene Ther 2014; 25:529-538.
26. Tesh R B, Peralta P H, Johnson K M. Ecologic studies of vesicular stomatitis virus. Am J Epidemiol 1969; 90:255-261.

27 Wilson J M, Flotte T R. Moving Forward After Two Deaths in a Gene Therapy Trial of Myotubular Myopathy. Hum Gene Ther 2020; 31:695-696.
28. Henkel A S, Kavesh M H, Kriss M S, Dewey A M, Rinella M E, Green R M. Hepatic Overexpression of Abcb11 Promotes Hypercholesterolemia and Obesity in Mice. Gastroenterology 2011; 141:1404-1411.e2.
29. Hansson M D, Rzeznicka K, Rosenback M, Hansson M, Sirijovski N. PCR-mediated deletion of plasmid DNA. Anal Biochem 2008; 375:373-375.
30. Shin A C, Fasshauer M, Filatova N, et al. Brain insulin lowers circulating BCAA levels by inducing hepatic BCAA catabolism. Cell Metab. 2014; 20 (5): 898-909. doi: 10.1016/j.cmet.2014.09.003.
31. Homanics G E, Skvorak K, Ferguson C, Watkins S, Paul H S. Production and characterization of murine models of classic and intermediate maple syrup urine disease. BMC Med Genet. 2006; 7:33. Published 2006 Mar. 31. doi: 10.1186/1471-2350-7-33.
32. Silver G, Mercimek-Andrews S. Inherited Metabolic Disorders Presenting with Ataxia. Int J Mol Sci. 2020; 21 (15): 5519. Published 2020 Aug. 1. doi: 10.3390/ijms21155519
33. Smith D, Wallom K L, Williams I M, Jeyakumar M, Platt F M. Beneficial effects of anti-inflammatory therapy in a mouse model of Niemann-Pick disease type C1. Neurobiol Dis. 2009; 36 (2): 242-251. doi: 10.1016/j.nbd.2009.07.010.

1. A retroviral vector comprising a 5′ long terminal repeat (LTR), a primer binding site, a heterologous intron, a transgene insertion site, and a 3′ LTR, wherein the heterologous intron is located immediately downstream of the 5′ LTR.

2. The retroviral vector of claim 1, wherein the primer binding site (PBS) is located between the 5′ LTR and the intron.

3. The retroviral vector of claim 1, wherein the 5′ LTR comprises an HIV-1 LTR or derivative sequence thereof.

4. The retroviral vector of claim 3, wherein the 5′ LTR has the sequence of SEQ ID No. 2 or a derivative sequence having at least about 40% identity thereof.

5. The retroviral vector of claim 1, wherein the 3′ LTR comprises a mutated HIV-1 LTR or derivative sequence thereof, optionally wherein the 3′ LTR has the sequence of SEQ ID No. 8 or a derivative sequence having at least about 40% identity thereof.

6. (canceled)

7. The retroviral vector of claim 5, wherein the 3′ LTR comprises a polyadenylation (polyA) site in an R domain and/or a HIV-1 transacting responsive (TAR) hairpin site.

8. The retroviral vector of claim 1, wherein the heterologous intron comprises a sequence of 100 to 2,000 base pairs, optionally wherein the heterologous intron comprises an intron from the human elongation factor 1a gene or an intron from the human β-globin gene.

9. (canceled)

10. The retroviral vector of claim 1, wherein the vector further comprises an HIV-1 psi packaging sequence and Rev Response Element (RRE) located downstream of the 3′ LTR.

11. The retroviral vector of claim 1, further comprising an expressible transgene in the transgene insertion site.

12. The retroviral vector of claim 1, wherein the vector further comprises a post-transcriptional regulatory element (PRE).

13. The retroviral vector of claim 1, wherein PRE is located between the transgene insertion site and the 3′ LTR.

14. The retroviral vector of claim 1, wherein the vector further comprises a promoter to drive transcription of the vector genome.

15. The retroviral vector of claim 1, wherein the vector is derived from a lentivirus.

16. A host cell or virion containing the vector of claim 1.

17-19. (canceled)

20. A pharmaceutical composition comprising the vector of claim 1 or a virion comprising the vector of claim 1.

21. The pharmaceutical composition of claim 20, wherein the composition is formulated for intravenous infusion.

22. The pharmaceutical composition of claim 20, wherein the composition is co-administered with an immunosuppressant.

23. (canceled)

24. A method of delivering a gene to a target cell, the method comprising administering an effective amount of the vector of claim 1 or a virion comprising the vector of claim 1 to the target cell, wherein the vector comprises an expressible transgene.

25. The method of claim 24, wherein the vector is co-transduced with an engineered Tat plasmid to enhance expression efficiency.

26. A cell or transgenic animal produced by the method of claim 24.

27. (canceled)


SEQUENCE LISTING (14 sequences)

SEQ ID NO: 1 - DNA, 597 bp, Human cytomegalovirus
  source 1..597: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaacc

SEQ ID NO: 2 - DNA, 181 bp, Human immunodeficiency virus 1
  source 1..181: mol_type = genomic DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 3 - DNA, 18 bp, Human immunodeficiency virus 1
  source 1..18: mol_type = genomic DNA
tggcgcccgaacagggac

SEQ ID NO: 4 - DNA, 939 bp, Homo sapiens
  source 1..939: mol_type = genomic DNA
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 5 - DNA, 476 bp, Homo sapiens
  source 1..476: mol_type = genomic DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 6 - DNA, 118 bp, Human immunodeficiency virus 1
  source 1..118: mol_type = genomic DNA
ttttaaaagaaaaggggggattggggggtacagtgcaggggaaagaatagtagacataatagcaacagacatacaaactaaagaattacaaaaacaaattacaaaaattcaaaatttt

SEQ ID NO: 7 - DNA, 589 bp, Woodchuck hepatitis virus
  source 1..589: mol_type = genomic DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 8 - DNA, 234 bp, Human immunodeficiency virus 1
  source 1..234: mol_type = genomic DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 9 - DNA, 234 bp, Human immunodeficiency virus 1
  source 1..234: mol_type = genomic DNA
tggaagggctaattcactcccaacgaagacaagatctgctttttgcttgtactgggtctctctggttagaccagatctgagcctgggagctctctggctaactagggaacccactgcttaagcctcaacaaagcttgccttgagtgcttcaagtagtgtgtgcccgtctgttgtgtgactctggtaactagagatccctcagacccttttagtcagtgtggaaaatctctagca

SEQ ID NO: 10 - DNA, 234 bp, Human immunodeficiency virus 1
  source 1..234: mol_type = genomic DNA
tggaagggctaattcactcccaacgaagacaagatctgctttttgcttgtactgggtctctctggttagaccagatctgagcctgggagttctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 11 - DNA, 234 bp, Human immunodeficiency virus 1
  source 1..234: mol_type = genomic DNA
tggaagggctaattcactcccaacgaagacaagatctgctttttgcttgtactgggtctctctggttagaccagatctgagcctgggagttctctggctaactagggaacccactgcttaagcctcaacaaagcttgccttgagtgcttcaagtagtgtgtgcccgtctgttgtgtgactctggtaactagagatccctcagacccttttagtcagtgtggaaaatctctagca

SEQ ID NO: 12 - DNA, 126 bp, Human immunodeficiency virus 1
  source 1..126: mol_type = genomic DNA
ctctctcgacgcaggactcggcttgctgaagcgcgcacggcaagaggcgaggggcggcgactggtgagtacgccaaaaattttgactagcggaggctagaaggagagagatgggtgcgagagcgtc

SEQ ID NO: 13 - DNA, 234 bp, Human immunodeficiency virus 1
  source 1..234: mol_type = genomic DNA
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 14 - DNA, 1324 bp, Human immunodeficiency virus 1
  source 1..1324: mol_type = genomic DNA
ctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaac